(19) United States
(12) Patent Application Publication
(10) Pub. No.: US 2025/0345529 A1
(43) Pub. Date: Nov. 13, 2025

(54) SYSTEM AND METHOD OF DETECTING AND MANAGING BUBBLES IN FLUID LINES

(71) Applicant: HYBERNIA MEDICAL, LLC, New Rochelle, NY (US)

(72) Inventors: Jae H Choi, Jericho, NY (US); John Pile-Spellman, New Rochelle, NY (US); Vincent N Sullivan, Palo Alto, CA (US); Jennifer Hodor, Sunnyvale, CA (US); Jason John Umhoefer, Redwood City, CA (US); Jerome Jackson, Los Altos, CA (US)

(73) Assignee: HYBERNIA MEDICAL, LLC, New Rochelle, NY (US)

(21) Appl. No.: 19/200,986

(22) Filed: May 7, 2025

Related U.S. Application Data
(60) Provisional application No. 63/643,684, filed on May 7, 2024.

(51) Int. Cl. A61M 5/36; F04B 49/00; F04B 51/00

(52) U.S. Cl. CPC A61M 5/36; F04B 49/00; F04B 51/00

(57) ABSTRACT

A system for managing air bubbles in fluid lines includes a pump, valves, a bubble chamber, sensors, and a controller unit. The pump pulls fluid from a reservoir, valves create a dual pressure network, and the bubble chamber captures air bubbles. Sensors measure physical properties and the controller unit receives input from these sensors to control system components.

### BACKGROUND OF THE INVENTION

In applications where fluid is delivered through a tube, such as a vascular infusion line, it can be important to avoid inadvertent delivery of bubbles. This is especially critical for clinical applications where a therapeutic fluid is delivered into the blood stream. Furthermore, whereas bubbles that are inadvertently delivered with intra-venous fluid delivery may be cleared through physiological gas exchange in the lung, bubbles in intra-arterial delivery have a high risk of forming gas emboli that are carried into the perfused tissue or organ and cause embolic infarcts.

In industrial, laboratory, and a growing number of clinical applications (especially endovascular and intra-arterial applications), inadvertent delivery of fluids that are contaminated with bubbles is undesirable. For instance, bubbles may lead to fabrication of products that are defective or of low quality, lead to laboratory results that are false and uninterpretable, and may cause embolic tissue infarcts in humans that result in disability or death. The present invention describes a system and method of detecting bubbles in fluid delivery lines and managing bubbles that are inadvertently introduced into fluid delivery lines. Such inadvertent introduction of bubbles may occur from cracks and damages along the fluid delivery path through which a connection is established between ambient air and intraluminal space of the fluid delivery path. Such inadvertent introduction of bubbles may also occur during processes, when via an external system fluid is added to or removed from the fluid delivery path and that external system is contaminated with bubbles or when such processes allow ambient gas to be introduced into the fluid delivery path.

### SUMMARY OF THE INVENTION

The claimed technique detects bubbles in fluid path by measuring intraluminal pressure relative to the force or displacement applied to the fluid in the fluid path and analyzing pressure changes (amplitudes) over time and analyzing pressure wave patterns over time. In one embodiment, the system may manage bubbles that enter the fluid path by placing an in-line bubble chamber to trap the bubbles before bubbles can travel downstream along the pressure gradient. Bubbles that enter the fluid path may be advantageously managed by creating a positive intraluminal pressure and avoid ambient gas from entering the fluid path. In one embodiment, the system may be composed as a disposable cartridge that comprises a pump fluid chamber (cassette), fluid path, bubble chamber, pressure transducer, and electrical connections, that is inserted into a pump-controller unit.

A system for managing air bubbles in fluid lines includes a pump exerting a force to pull a fluid from a fluid reservoir through a first fluid path and one or more valves separating a second fluid path from the first fluid path thereby creating a dual pressure network. Further down the network is a bubble chamber integrated into a proximal position of the second fluid path. The system may further include one or more sensors detecting measurements of physical properties across the network. According to one embodiment, a first sensor integrated into a distal position of the second fluid path wherein the first sensor is a pressure transducer. The system may further include a controller unit configured to receive input from the first sensor and control one or more system components, based, at least in part on the received input.

According to one embodiment, the pump is a piston pump although other types of pumps may be used. The fluid reservoir may be located advantageously in a position above the pump. The bubble chamber may be in further communication with a vent drain inlet wherein the vent drain inlet is positioned against gravity and wherein the bubble chamber includes a bubble chamber outlet positioned towards gravity. A bubble chamber inlet may also positioned above the bubble chamber according to a gravitational axis. According to one embodiment, the one or more system components the controller is configured to control includes the pump. The controller may receive input from a second sensor wherein a second sensor obtains a pressure measurement associated with the first fluid path. The controller may further receive input from a third sensor wherein the third sensor obtains a volume measurement associated with the pump. According to one embodiment, the controller conducts a fluid analysis the input received from the pressure transducer, the pressure measurement associated with the first fluid path and the volume measurement. The controller may transmit commands to the one or more system components on the network based on the fluid analysis.

A method for managing air bubbles in fluid lines may comprise exerting a force via a pump to pull a fluid from a fluid reservoir through a first fluid path and creating a dual pressure network by separating a second fluid path via one or more valves from the first fluid path. The method may further include integrating a bubble chamber for capturing one or more air bubbles wherein the bubble chamber is located in a proximal position of the second fluid path and integrating a pressure transducer into a distal position of the second fluid path. According to one embodiment, the method may include configuring a controller unit to receive input from the pressure transducer and control one or more system components, based, at least in part on the received input.

The method of described above may utilize a piston pump and a fluid reservoir may be located in a position above the pump. The bubble chamber may be placed in communication with a vent drain inlet wherein the vent drain inlet is positioned against gravity and wherein the bubble chamber includes a bubble chamber outlet positioned towards gravity. According to one embodiment, the bubble chamber inlet is positioned above the bubble chamber according to a gravitational axis. The controller may also be configured to control the pump and receive input from a second sensor wherein a second sensor obtains a pressure measurement associated with the first fluid path. The controller may also receive input from a third sensor wherein the third sensor obtains a volume measurement associated with the pump. According to one embodiment, the controller conducts a fluid analysis the input received from the pressure transducer, the pressure measurement associated with the first fluid path and the volume measurement. The controller may also transmit commands to the one or more system components on the network based on the fluid analysis.

### BRIEF DESCRIPTION OF THE FIGURES

FIG. 1 illustrates a system for managing and detecting bubbles in fluid lines according to one embodiment.

FIG. 2 illustrates a system for managing and detecting bubbles in fluid lines according to one embodiment.

FIG. 3 illustrates a graph of pressure over time.

FIG. 4 illustrates a graph of pressure over time.

FIG. 5 illustrates a system for managing and detecting bubbles in fluid lines according to one embodiment.

FIG. 6 illustrates a system for managing and detecting bubbles in fluid lines according to one embodiment.

FIG. 7 illustrates a system for managing and detecting bubbles in fluid lines according to one embodiment.

FIG. 8 illustrates a system for managing and detecting bubbles in fluid lines according to one embodiment.

FIG. 9 illustrates two graphs of pressure over time.

FIG. 10 illustrates a system for managing and detecting bubbles in fluid lines according to one embodiment.

FIG. 11 illustrates a system for managing and detecting bubbles in fluid lines according to one embodiment.

FIG. 12 illustrates a system for managing and detecting bubbles in fluid lines according to one embodiment.

FIG. 13 illustrates a graph for the pressure in a two piston pump according to one embodiment.

FIG. 14. illustrates a graph showing average pressure over time intervals.

### DETAILED DESCRIPTION OF THE INVENTION

The present invention is described in the following examples, which are set forth to aid in the understanding of the invention, and should not be construed to limit in any way the scope of the invention as defined in the claims which follow thereafter.

The present System and Method shall be described and exemplified with a clinical endovascular infusion system where a simple or Newtonian fluid is delivered. However, this invention can be applied to other applications in industry and laboratory practice where inadvertent delivery of bubbles in fluid-carrying lines are undesirable.

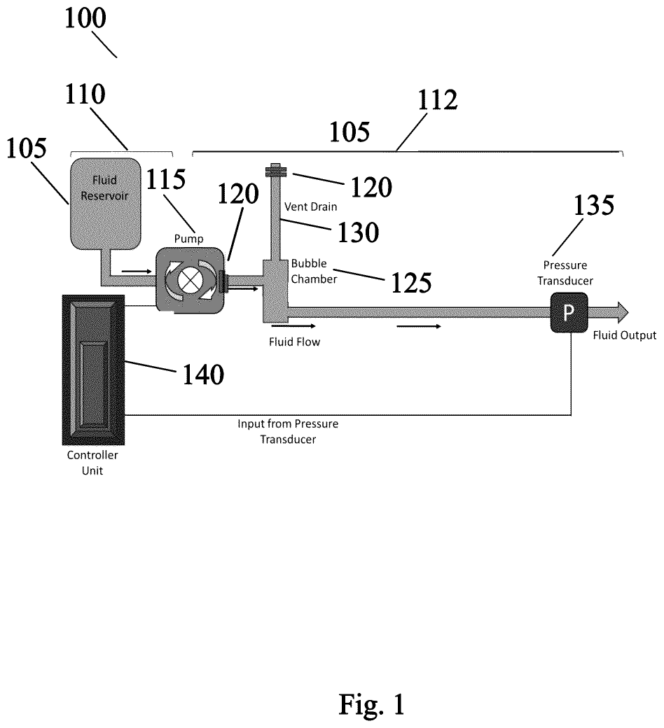

FIG. 1 illustrates a system 100 for managing and detecting bubbles in fluid lines according to one embodiment. In one embodiment, the System may comprise a fluid reservoir 105 (e.g. physiological fluid), fluid path 110, 112 (e.g. fluid line and endovascular catheter), a pump 115 (e.g. that drives a piston mechanism), valves 120, bubble chamber 125, bubble chamber vent drain 130, pressure transducer 135, and electrical controller unit 135 with software (e.g. computer).

The fluid reservoir 105 in this system serves as the starting point for fluid delivery. It is typically a sealed or semi-sealed container, positioned at the upstream end of the system, designed to store and supply fluid including, but not limited to, medication, saline solution, or nutrient fluid for controlled injection. In other embodiments, the fluid reservoir may hold other types of fluids with industrial applicability.

As shown in FIG. 1, the fluid reservoir may be connected directly to the inlet of the pump, allowing fluid to be drawn in either by gravity or pressure. The fluid reservoir may be made from biocompatible materials, like medical-grade plastic or glass, to ensure sterility and prevent chemical interaction with the stored liquid. Depending on the application, it may be equipped with features such as a vented cap, a level sensor, or filters to prevent contamination and allow pressure equalization. It may also be positioned at a slightly elevated level to help maintain positive pressure at the pump inlet and reduce the chance of air bubbles entering the line.

According to one embodiment, the pump exerts a negative force to pull the fluid from the reservoir (Fluid Path 1) and a positive force to push the fluid downstream through the tubing (Fluid Path 2). The pump may include or be comprised of one or more peristaltic pumps, where fluid is pushed through flexible tubing by rollers compressing it in a rotating motion. The advantages peristaltic pumps are their sterility in the sense that the fluid only contacts the tubing and that they provide a smooth pulseless flow. Peristaltic pumps may be utilized in infusion pumps, dialysis machines, fluid transfer systems and the like.

The pump may also include or be comprised of one or more syringe pumps where a motor pushes or pulls the plunger of a syringe with precise control. Syringe pumps are typically highly accurate for small-volume dosing and are ideal for slow, continuous injections, such as drug delivery, microfluidics, high-precision research and the like. The pump may also include or be comprised of one or more gear pumps where interlocking gears move fluid in a steady, enclosed flow. Gear pumps are typically used for high pressure and flow rates. The pump may also include or be comprised of one or more diaphragm pumps, where a diaphragm flexes to create suction and discharge cycles. Diaphragm pumps are advantageous for air-free flow and can handle particulates or corrosive fluids with use cases in sampling, filtration, or sensitive reagent delivery. The pump may also include or be comprised of one or more piston pumps, where a piston mechanically pushes fluid through a chamber. The piston pumps are ideal for high precision, repeatability, and high-pressure applications. The pump may also include or be comprised of one or more microfluidic Pumps (MEMS-based), where miniature mechanical or electrokinetic mechanisms drive fluid through tiny channels. Microfluidic pumps are typically ultra-precise, and compact and are suitable for lab-on-a-chip or portable devices.

According to one embodiment, valves separate the two fluid path systems and create two separate pressure systems, the low or negative pressure first fluid path and the positive pressure second fluid path. The bubble chamber is integrated into the proximal part of the second fluid path, whereas the pressure transducer is located downstream of the second fluid path. The bubble chamber is oriented in a way that the vent drain inlet is directed against gravity and the outlet of bubble chamber (downstream of the second fluid path) is positioned towards gravity. With that, the bubble chamber inlet is positioned above the bubble chamber outlet in the gravitational axis. A pressure transducer 135 (or pressure sensor) is a device that measures pressure in a system and converts that measurement into an electrical signal that can be read and interpreted by a controller or monitoring system.

The controller unit in this system acts as the central brain, orchestrating the operation of components involved in fluid delivery. In one embodiment, the controller unit includes microcontroller or embedded processor for executing logic, processing inputs and sending commands to hardware components. The controller unit may further include a user interface which may include an touchscreen display, a keypad, a plurality of buttons or knobs and indicator LEDs. The controller may also include a combination of digital and analog input and outputs to communicate with sensors, actuators and pumps. In some embodiments the controller may include built-in safety logic to detect and respond to overpressure, air bubbles, occlusions, leaks and pump malfunctions. The controller may further include one or more communication ports for data exchange like USB and/or ethernet or Wi-Fi for remote monitoring and/or control.

As an electronic module, the controller may monitor and regulate key parameters such as flow rate, pump speed, pressure, and valve positions to ensure precise and safe administration of fluids. At its core, the controller interfaces with the pump, receiving programmed instructions or real-time user input to start, stop, or modulate fluid flow. As noted above, it may include digital display screens, control buttons, or touch interfaces to allow clinicians or technicians to set dosage volumes, timing intervals, and safety thresholds.

According to one embodiment, the controller may manage feedback loops using data from sensors placed throughout the system. For example, pressure sensors might alert the controller to a blockage, or air detectors might trigger a shutoff to prevent bubbles from reaching the patient. The controller can also communicate with the degassing system, activating a vacuum or triggering alerts if gas is not being effectively removed. Depending on the application, the controller may store usage logs, event history, and calibration data, supporting both quality control and regulatory compliance. In medical environments, it might also integrate with hospital networks for remote monitoring or electronic health records. The controller unit may include controller software that controls the pump mechanism (e.g. driving the piston action, turning pump ON and OFF) and receives input from the pressure transducer. The controller unit may also receive input from a force meter or displacement meter that is installed in the pump unit.

The controller 135 may be configured with software to accomplish a variety of tasks. According to one embodiment, the controller may be configured to perform air detection and leak detection. The controller may also be configured to conduct periodic checks, routine or scheduled checks. The controller may be pre-programmed with modifiable algorithms to receive input, analyze the input and deliver an output which may be in the form of commands to other components of the system and/or reporting or logging the input along with the analysis. For example, from a defined starting position (e.g. “home”) the rate of increase in pressure as the pump speed increases from “drip” to 70 ml/min (e.g. at the beginning of an auto flush). This rate can be used to detect air in the line (between the cartridge and the catheter). According to one embodiment, the desired algorithm may include the following steps 1) move the pump motor to the home position (speed profile can remain enabled) 2) set speed to drip for 0.5 s then rapidly ramp speed to 70 ml/min and 3) monitor the pressure increase during the first Is of the ramp and compare the rise against a prescribed air detection threshold. The presence of air may significantly slow the pressure rise as pump speed increases. The preceding air detection algorithm does not displace any leak detection algorithm used with the claimed system. In fact, the leak detection may continue to be checked. The system may also continue and contemporaneously check for occlusion.

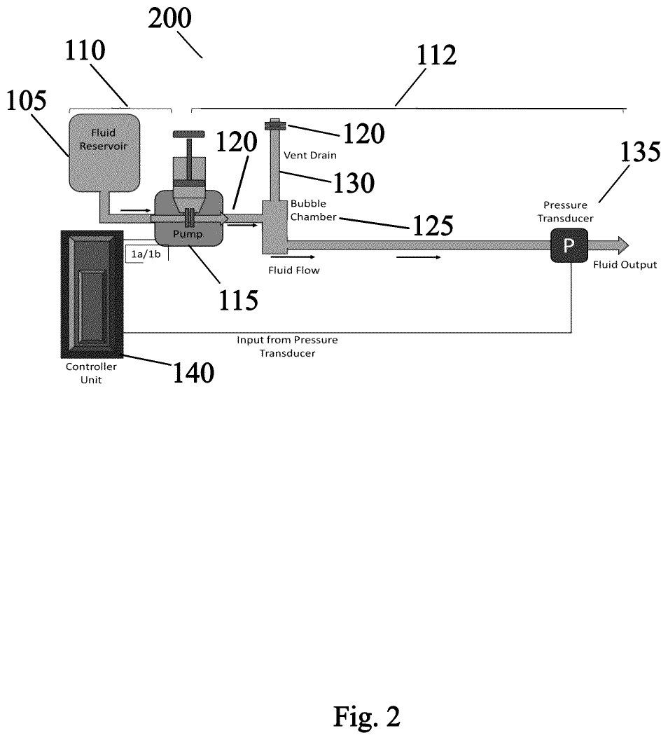

FIG. 2 illustrates a system 200 for managing and detecting bubbles in fluid lines according to one embodiment. As shown in FIG. 2, a pump delivers fluid through a fluid line or tube by applying a displacing force to the fluid in the line. According to Poiseuille, Equation 1: the flow (Q) is proportional to the pressure difference (ΔP), tube radius to the power of 4 (r4), 1/viscosity (1/η) and 1/tube length (1/L).

$$Q = \frac{π Δ P {r}^{4}}{8 η L} \qquad Equation 1$$

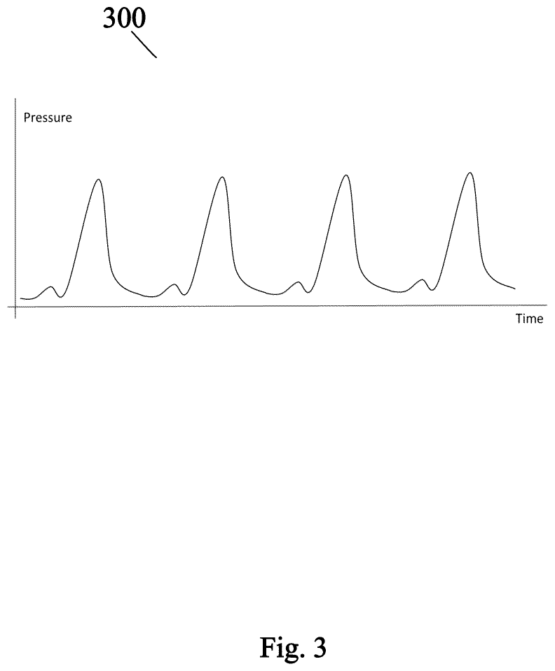

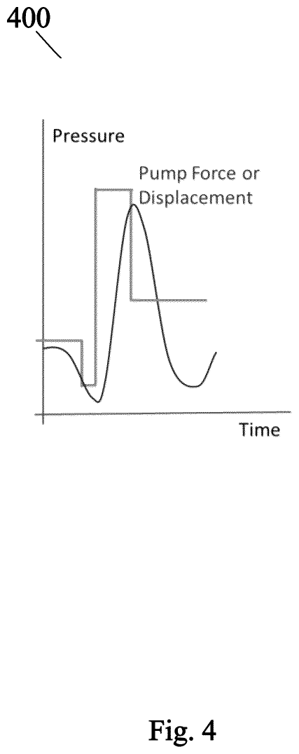

This means, that, in a given fluid delivery system with known material parameters, the intraluminal pressure changes (over time) that are measured at a defined location can be known. For instance, this intraluminal pressure can be measured with an in-line pressure transducer. If this fluid delivery system is intact, the pressure changes can be predicted relative to the force or displacement applied to the fluid by the pump (normal pressure pattern, shown in FIGS. 3 and 4).

FIG. 3 illustrates a graph 300 of pressure over time. More specifically, graph 300 shows the rhythmic change in pressure over time. FIG. 4 illustrates a graph 400 of pressure over time. More specifically, graph 400 shows the rhythmic change in pressure over time overlayed with pump force displacement.

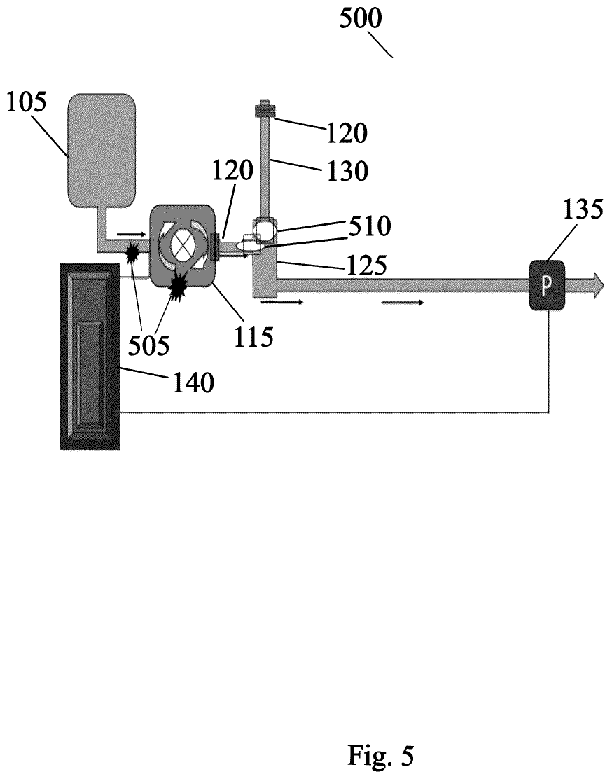

FIG. 5 illustrates a system 500 for managing and detecting bubbles in fluid lines according to one embodiment. FIG. 5 shows damage 505 in the fluid systems and communication with extraluminal space. For example, Gas may enter the fluid system through a damage 505 in the wall of the tubing or the pump. For instance, ambient air may enter through the leak and create bubbles that may persist if undissolved or even accumulate to form large bubbles 510 or enter in greater volumes into the fluid system. Gas may also be introduced by injection, e.g. injection of a drug (with a syringe that is contaminated with bubbles) into a fluid line.

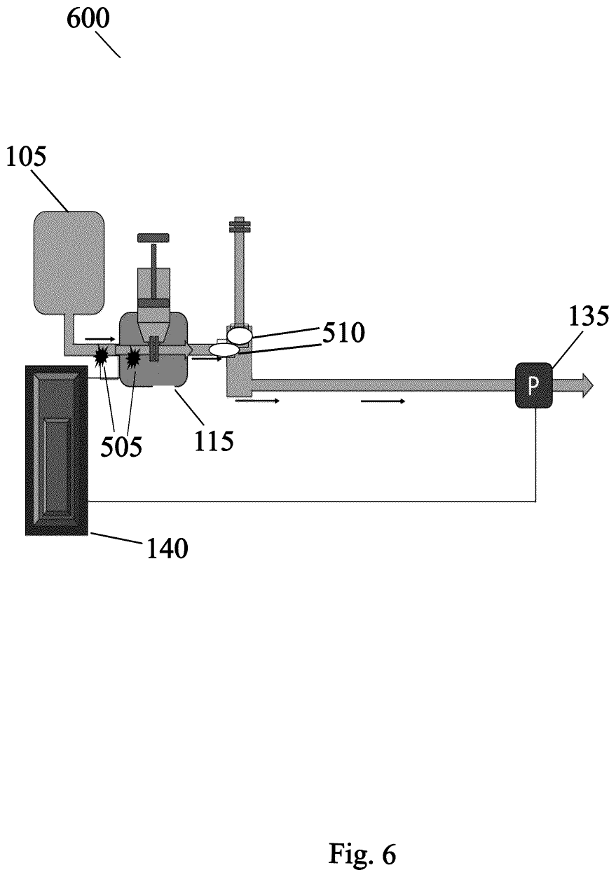

FIG. 6 illustrates a system 600 for managing and detecting bubbles in fluid lines according to one embodiment. Similar to FIG. 5, FIG. 6 illustrates damage 505 in the fluid systems along the first fluid path as well as the pump. Near the bubble chamber exists at least two bubbles 510.

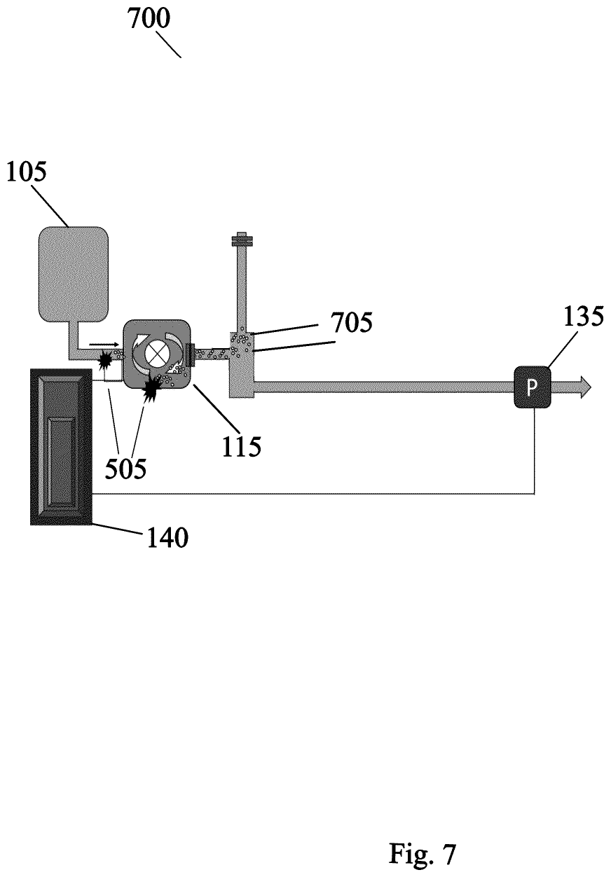

FIG. 7 illustrates a system 700 for managing and detecting bubbles in fluid lines according to one embodiment. The system 700 shows damage 505 to the first fluid path and the development of small bubbles in three locations. The first location is along the first fluid path. The second location is in the pump. The third location of small bubbles 705 is in the bubble chamber. As can be seen in FIG. 7, the damage 505 can introduce air bubbles into the fluid system.

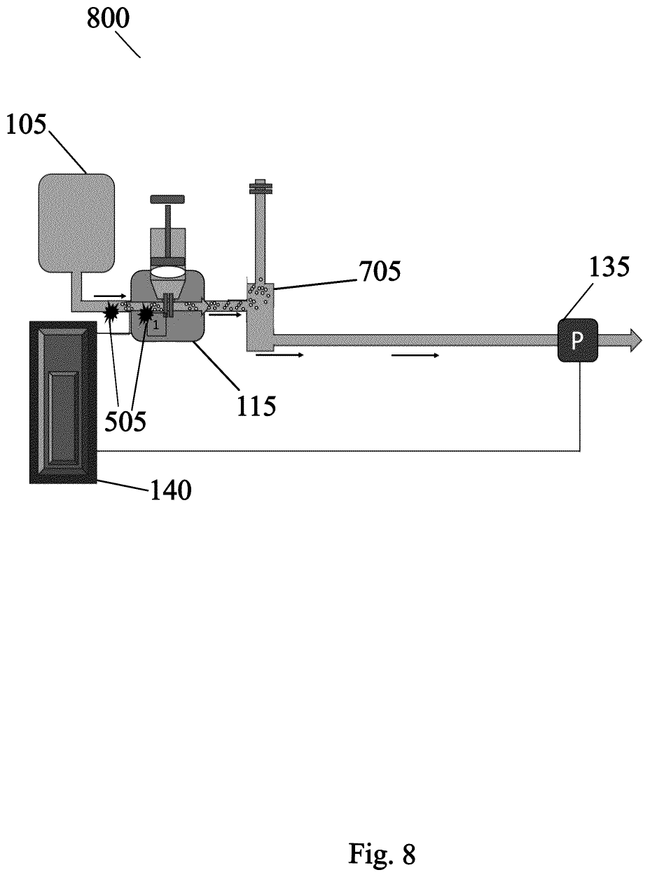

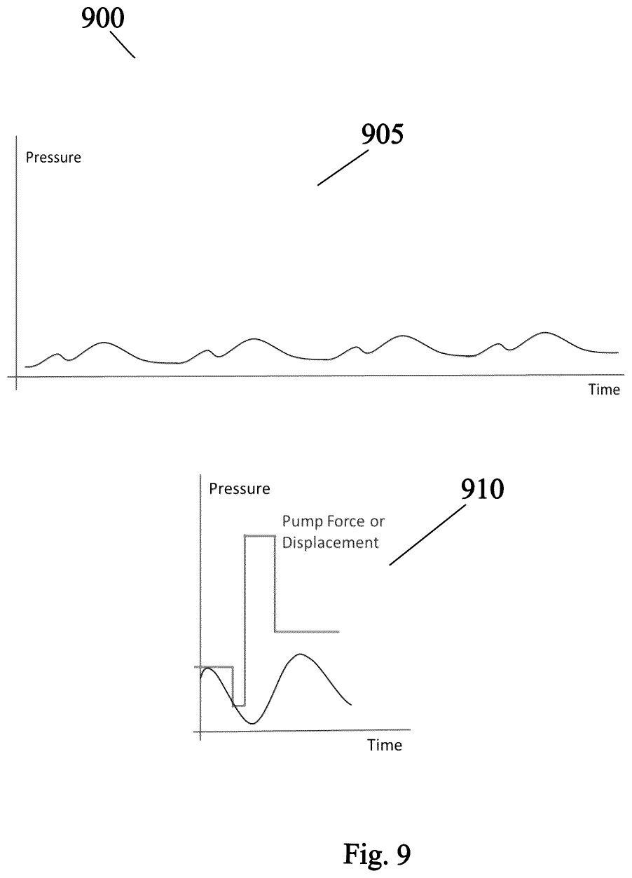

FIG. 8 illustrates a system 800 for managing and detecting bubbles in fluid lines according to one embodiment. Similar to FIG. 7, the system 800 shows damage 505 to the first fluid path and the development of small bubbles in three locations. The first location is along the first fluid path. The second location is in the pump. The third location of small bubbles 705 is in the bubble chamber. As can be seen in FIG. 8, the damage 505 can introduce air bubbles into the fluid system. If gas enters the fluid line (e.g., ambient air through a leak in the pump or proximal fluid system) that is sufficient in volume to remain large and undissolved, this will alter the pressure pattern and, for instance, lead to a depressed pressure curve compared to the normal pressure pattern (Shown in FIG. 9). This abnormal pressure pattern is recognized by the controller which may trigger an alarm and turn off the pump.

FIG. 9 illustrates two graphs 900 of pressure over time. FIG. 9 shows a first graph 905 that reveals a depressed pressure curve over time. Depending the sensitivity of the controller for recognizing deltas in pressure patterns, this change in pressure can be recognized by the controller and result in the execution of commands directed to one or more components, e.g., the pump, fluid reservoir, and/or the like. A second graph 910 also shows a depressed pressure reading over time against pump force or displacement. If gas enters the fluid line, e.g. ambient air through a leak in the pump or proximal fluid system, in small amounts and form microbubbles, this may not alter the pressure pattern. However, in either case, the bubbles will enter the bubble chamber and float up against gravity. When microbubbles accumulate into larger bubbles and float toward the vent drain, the bubbles may be removed by opening the valve of the vent drain. When bubbles accumulate in sufficient amounts, this will alter the pressure pattern and, for instance, lead to a depressed pressure curve compared to the normal pressure pattern which will be recognized by the controller.

If there is a damage in the walls of the bubble chamber or distal fluid system (Fluid Path 2), e.g. exposing the fluid system to ambient air, air is unlikely to enter the system due to the higher intraluminal pressure in the distal fluid system compared to ambient air pressure.

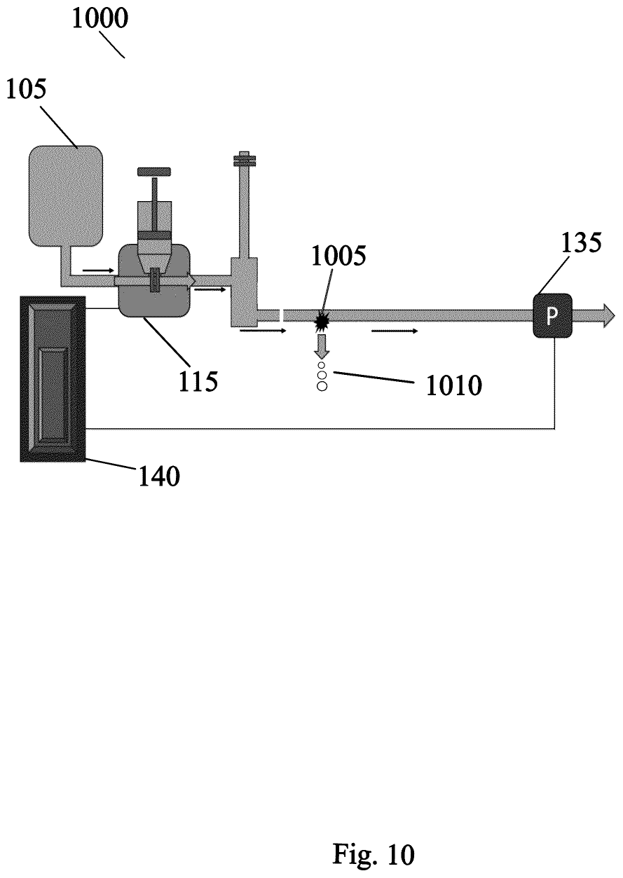

FIG. 10 illustrates a system 1000 for managing and detecting bubbles in fluid lines according to one embodiment. FIG. 10 shows damage 1005 on the second fluid path. The system further shows fluid exiting the second fluid path 1010 though the damage 1005 (leak).

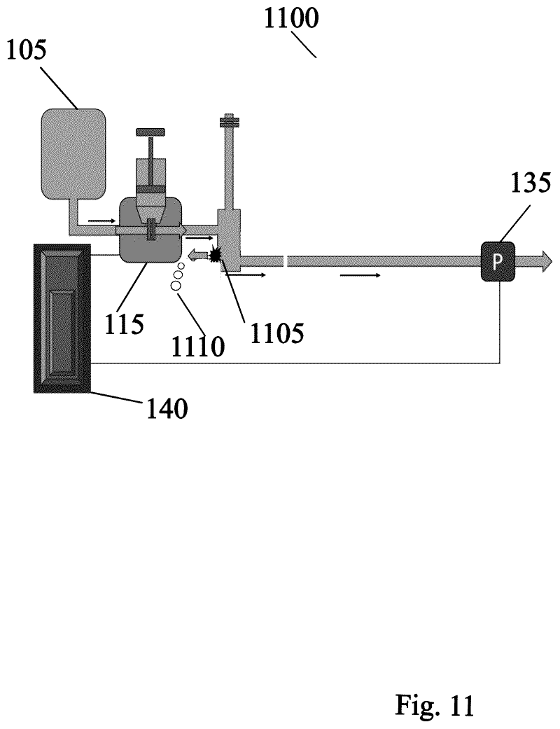

FIG. 11 illustrates a system 1100 for managing and detecting bubbles in fluid lines according to one embodiment. FIG. 11 shows damage 1105 on the bubble chamber. The system further shows fluid exiting the second fluid path 1110 though the damage 1105 (leak).

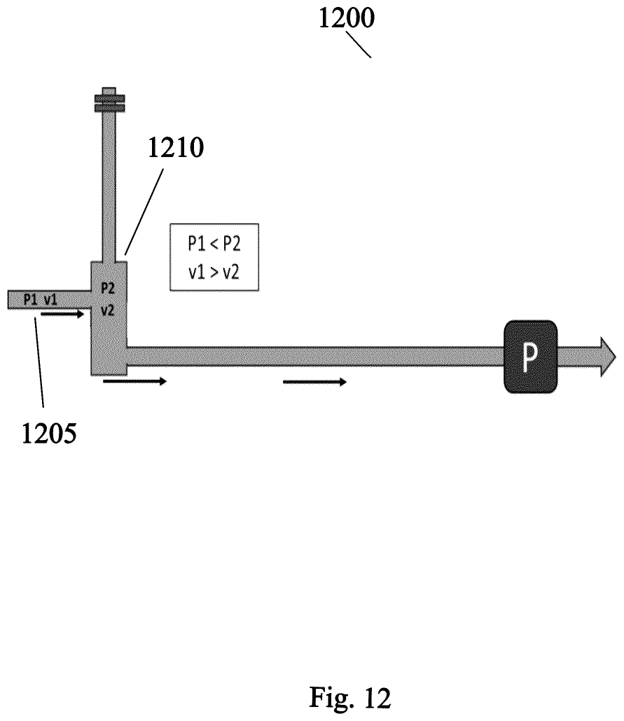

FIG. 12 illustrates a system 1200 for managing and detecting bubbles in fluid lines according to one embodiment. According to the Bernoulli's Principle, the intraluminal pressure inside the (wider) bubble chamber (P2/V2) 1210 is higher than the pressure in the (narrower) preceding fluid line (P1/V1) 1205, which will also result in fluid exiting the system through the damage in the wall. Either case will alter the pressure pattern and, for instance, lead to a depressed pressure curve compared to the normal pressure pattern. This abnormal pressure pattern is recognized by the controller which may trigger an alarm and turn off the pump.

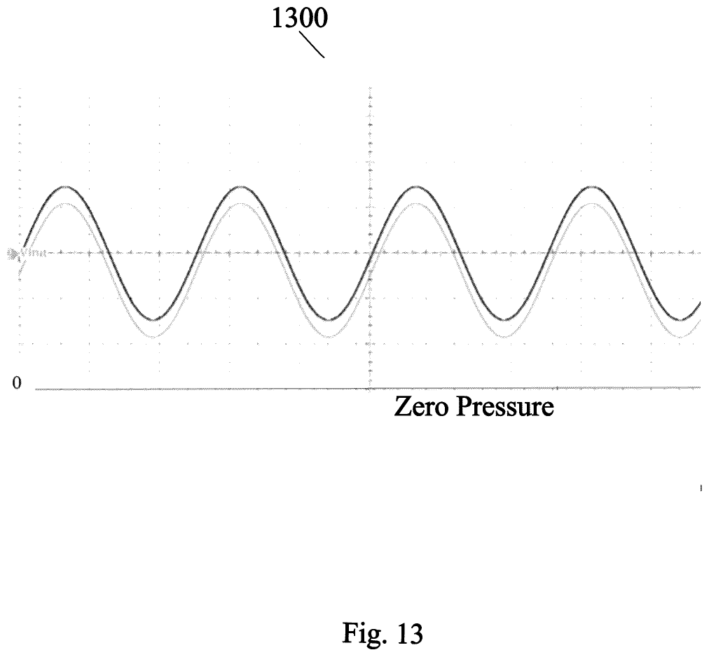

FIG. 13 illustrates a graph 1300 for the pressure in a two piston pump according to one embodiment. As illustrate the graph illustrates the an X and a Y axis where the X axis represents time and the Y axis represents the value of pressure. Although embodiments of the claimed system may incorporate a single piston pump, a single piston pump may develop a negative pressure during part of their cycle, that can aspirate air if any leak develops. In one embodiment, the two-piston pump prevents the ingress of bubbles on the high pressure side because of the continuous positive pressure. The pressure output is approximately 100-150 psi on the low pressure output and 250-400 psi on the high pressure output curve. Thus, air cannot be aspirated even if there is damage to the cassette or tubing on the high pressure side of the pumping network.

Depending on the material composition, fluid lines and catheters can have different compliance characteristics. The compliance characteristics can affect the pressure wave during the pumping action which can be measured and be known. In one embodiment of the system, the controller can “learn” during the initial set up under prescribed conditions the distinct compliance and pressure characteristics. This is done by executing a series of different pump actions or maneuvers and thereby creating different pressure waves (i.e. varying the velocity and duration of the injection force) and measuring the resulting pressure patterns which are inputted into the controller and analyzed. As such, besides constant monitoring of the force/pressure observed vs. expected over the duration of the infusion, the most sensitive of these maneuvers may be repeated at intervals to detect physical changes to the fluid such as contamination with bubbles. When additional confidence is required, known amounts of air can be introduced, and the force/pressure curves can be analyzed to determine the detection thresholds.

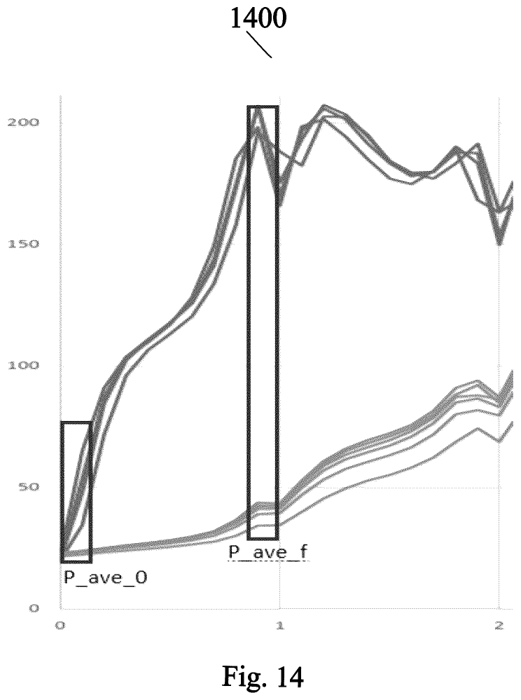

FIG. 14. illustrates a graph 1400 showing average pressure over time intervals. FIG. 14 shows P_ave_0 which is the average of five samples taken at 20 ms intervals from T=20 to 100 ms. FIG. 14 also shows P_ave_f which is the average of five samples taken at 20 ms intervals from T=900 ms to 980 ms. To understand the graph we should understand the ability of the controller. For example, the Controller may be configured with an auto flush mode or algorithm. When Auto Flush is pressed, the system may be directed to the home position. From there, the controller may send SPI command 0x0622 to the SAMM Pump FPGA to enable the Home Stop. The speed profile may remain enabled. The controller may then set speed=200 DAC counts (assumes the software ramps to this speed from drip). The controller may monitor (poll) the 0x0022 cycle count register to determine when the pump has reached the home position (stops incrementing for >10 ms). Once home, the controller may send SPI 0x0623 to the SAMM Pump FPGA to Disable the Home Stop.

Next, the controller may set speed to drip. Then the controller will wait 500 ms. At “t=0”, ramp the speed rapidly to 70 ml/min (300 DAC counts). Then average the first 5 pressure samples, taken each 20 ms from t=20 ms to t=100 ms and record this average as the initial pressure (P_ave_0) (Shown in FIG. 14) At t=900 ms, average 5 pressure samples, taken each 20 ms from t=900 to t=980 and record this average as the “final” pressure (P_ave_f). (Shown in FIG. 14). The controller may also compare (P_ave_f-P_ave_0) against the “Air detection threshold” (nominally 40 psi, although other thresholds may be used). If the difference is lower than the threshold, enter the Error state with the pressure-based air detection status. The controller may return pump speed to V_init, hold pump speed at V_init for 2000 ms and Re-enable temperature PID control of pump speed. It should be noted that “masking” of the graphed temperature (FIG. 14) should continue until PID control is re-enabled. At t=900 ms, average 5 pressure samples, taken each 20 ms from t=900 to t=980 and record this average as the “final” pressure (P_ave_f), shown in FIG. 14.

The system may continue Auto Flush (at 70 ml/min) for the remainder of the Auto Flush volume and compare (P_ave_f−P_ave_0) against the “Air detection threshold” (e.g., 40 psi). If the difference is lower than the threshold, the Controller may enter the Error state with the pressure-based air detection status. As noted above, the controller may be configured to conduct periodic checks during a treatment. In one embodiment, the controller may keep a 300 ms moving average of pressure (P_ave). Then the controller may save the current pump speed as “V_init.” The controller will then go to the home position and send SPI 0x0622 command to the SAMM Pump FPGA to Enable the Home Stop. According to one embodiment, the speed profile can remain enabled. For example, speed may be equal to 200 DAC counts (which assumes the software ramps to this speed it's present value). The controller may then monitor (poll) the 0x0022 cycle count register to determine when the pump has reached the home position (stops incrementing for >10 ms). Once home, the controller may send SPI 0x0623 command to the SAMM Pump FPGA to Disable the Home Stop. Next, the controller may set speed to drip and wait 500 ms. At “t=0”, the controller may ramp the speed rapidly to 70 ml/min (300 DAC counts). The controller may then average the first 5 pressure samples, taken each 20 ms from t=20 ms to t=100 ms and record this average as the initial pressure (P_ave_0). (Shown in FIG. 14). At t=900 ms, the controller may average 5 pressure samples, taken each 20 ms from t=900 to t=980 and record this average as the “final” pressure (P_ave_f). [see FIG. 14]. The controller may also compare (P_ave_f−P_ave_0) against the “Air detection threshold” (e.g., 40 psi). If the difference is lower than the threshold, enter the Error state with the pressure-based air detection status. The controller may then return pump speed to V_init, hold pump speed at V_init for 2000 ms (TBD) and re-enable Temperature PID control of pump speed. It should be noted that “masking” of the graphed temperature should continue until PID control may be re-enabled.

The distinguishing characteristics of the claimed technique include how the system uses detectors, types of measurements, the physical event detected, the scale of operation, dissolved gas sensitivity and upstream and downstream sensitivity. The focus here is on mechanical properties (e.g., pressure, volume changes) across the entire circuit, which gives the controller broader detection-including dissolved gas and air volume upstream/downstream.

The detectors include pressure and pump displacement sensors. The measurements taken may include force, volume and similar measurements. The physical event detected may include fluid and air compressibility. For example, the controller may track pressure changes and pump displacement (volume per stroke) to detect anomalies. In one embodiment, if the system detects that more displacement is required to achieve the same pressure, or that pressure builds too slowly or irregularly, it may infer the presence of air or a gas pocket in the line. This works over the entire circuit and can even detect dissolved gases because they influence compressibility as well.

While the foregoing invention has been described in some detail for purposes of clarity and understanding, it will be appreciated by one skilled in the art, from a reading of the disclosure, that various changes in form and detail can be made without departing from the true scope of the invention.

1. A system for managing air bubbles in fluid lines, comprising:
a pump exerting a force to pull a fluid from a fluid reservoir through a first fluid path;
one or more valves separating a second fluid path from the first fluid path thereby creating a dual pressure network;
a bubble chamber integrated into a proximal position of the second fluid path;
one or more sensors detecting measurements of physical properties across the network;
a first sensor integrated into a distal position of the second fluid path wherein the first sensor is a pressure transducer;
a controller unit configured to receive input from the first sensor and control one or more system components, based, at least in part on the received input.

2. The system of claim 1, wherein the pump is a piston pump.

3. The system of claim 1, wherein the fluid reservoir is located in a position above the pump.

4. The system of claim 1, wherein the bubble chamber in communication with a vent drain inlet wherein the vent drain inlet is positioned against gravity and wherein the bubble chamber includes a bubble chamber outlet positioned towards gravity.

5. The system of claim 4, wherein a bubble chamber inlet is positioned above the bubble chamber according to a gravitational axis.

6. The system of claim 1, wherein the one or more system components the controller is configured to control includes the pump.

7. The system of claim 1, wherein the controller receives input from a second sensor wherein a second sensor obtains a pressure measurement associated with the first fluid path.

8. The system of claim 7, wherein the controller receives input from a third sensor wherein the third sensor obtains a volume measurement associated with the pump.

9. The system of claim 8, wherein the controller conducts a fluid analysis the input received from the pressure transducer, the pressure measurement associated with the first fluid path and the volume measurement.

10. The system of claim 9, wherein the controller transmits commands to the one or more system components on the network based on the fluid analysis.

11. A method for managing air bubbles in fluid lines, comprising:
exerting a force via a pump to pull a fluid from a fluid reservoir through a first fluid path;
creating a dual pressure network by separating a second fluid path via one or more valves from the first fluid path;
integrating a bubble chamber for capturing one or more air bubbles wherein the bubble chamber is located in a proximal position of the second fluid path;
integrating a pressure transducer into a distal position of the second fluid path;
configuring a controller unit to receive input from the pressure transducer and control one or more system components, based, at least in part on the received input.

12. The system of claim 11, wherein the pump is a piston pump.

13. The system of claim 11, wherein the fluid reservoir is located in a position above the pump.

14. The system of claim 11, wherein the bubble chamber in communication with a vent drain inlet wherein the vent drain inlet is positioned against gravity and wherein the bubble chamber includes a bubble chamber outlet positioned towards gravity.

15. The system of claim 14, wherein a bubble chamber inlet is positioned above the bubble chamber according to a gravitational axis.

16. The system of claim 11, wherein the one or more system components the controller is configured to control includes the pump.

17. The system of claim 11, wherein the controller receives input from a second sensor wherein a second sensor obtains a pressure measurement associated with the first fluid path.

18. The system of claim 17, wherein the controller receives input from a third sensor wherein the third sensor obtains a volume measurement associated with the pump.

19. The system of claim 18, wherein the controller conducts a fluid analysis the input received from the pressure transducer, the pressure measurement associated with the first fluid path and the volume measurement.

20. The system of claim 19, wherein the controller transmits commands to the one or more system components on the network based on the fluid analysis.
